(19) United States
(12) Patent Application Publication
(10) Pub. No.: US 2002/0113939 A1
(43) Pub. Date: Aug. 22, 2002

(54) Ophthalmologic apparatus and displaying method for ophthalmic image

(76) Inventor: Takeshi Kitamura, Tochigi (JP)

Correspondence Address: FITZPATRICK CELLA HARPER & SCINTO, 30 ROCKEFELLER PLAZA, NEW YORK, NY, 10112, US

(21) Appl. No.: 09/955,187

(22) Filed: Sep. 19, 2001

(30) Foreign Application Priority Data
Feb. 22, 2001 (JP) 2001-047293

(51) Int. Cl. A61B 3/00; A61B 3/14

(52) U.S. Cl. 351/200

(57) ABSTRACT

An ophthalmologic apparatus has an image taking apparatus for image-taking an eye of a patient, a display for displaying an image of the eye of the patient, which is image-taken by the image taking apparatus, and a control device. The control device executes a first step of performing first image taking by the image taking apparatus, a second step of displaying a first image obtained in the first image taking on the display, a third step of performing second image taking by the image taking apparatus after the second step, and a fourth step of displaying a second image obtained in the second image taking together with the first image in a predetermined display layout on the display.

### BACKGROUND OF THE INVENTION

1. Field of the Invention

The present invention relates to an ophthalmologic apparatus such as an eye fundus camera or a medical filing apparatus, which is used in, for example, an ophthalmic hospital, and to a displaying method for an ophthalmic image.

2. Related Background Art

There has been an increase in a management form for managing an image of an eye of a patient, which is taken by an eye fundus camera, as an electronic file in a personal computer including a video capture board. Here, as a layout for displaying the taken image on a display, generally, thumbnail images reduced in an image taking order are arranged and displayed in order as shown in FIG. 12, or one taken image is singly displayed as shown in FIG. 13.

However, there are the following points to be improved for the above-mentioned examples.

(1) In a case of an image taking method of image-taking both the left and right eyes plural times at photofluorography or the like, image taking is performed while an eye of the patient to be image-taken is frequently changed between the left eye and the right eye. However, since the taken images are displayed in an image taking order, there is a case where it can not be judged whether the displayed image is the right eye or the left eye at first sight.

(2) When a wide area of an eye fundus is image-taken and paste processing is performed, an operator views an image and determines a position to be pasted for each image. Thus, the operation is very difficult.

(3) Recently, in order to judge diabetic retinopathy, the criterion in a case where a plurality of portions for the same eye are image-taken is proposed. However, since the determination of necessary image taking regions is left to an operator, the operator is burdened. Thus, the problems such as insufficient image taking and duplication of image taking of the same portion occur.

(4) A panorama display in which the taken images are superimposed on each other and a portion with a wide area can be viewed in a list form is allowed only after the image taking, and thus it cannot be checked during the image taking.

### SUMMARY OF THE INVENTION

A main object of the present invention is to provide a further improvement of a conventional ophthalmologic apparatus. One concrete object is to provide an ophthalmologic apparatus in which a display layout of an ophthalmic image displayed on a display is improved and superior usability is achieved.

Another object of the present invention is to provide a displaying method for an ophthalmic image, which can be determined through intuition by an operator.

To attain the above object, according to one aspect of the present invention, there is provided an ophthalmologic apparatus including:

an image taking apparatus for image-taking an eye of a patient;

a display for displaying an image of the eye of the patient, which is image-taken by the image taking apparatus; and

a control device,

wherein the control device executes;

a first step of performing first image taking by the image taking apparatus;

a second step of displaying a first image obtained in the first image taking on the display;

a third step of performing second image taking by the image taking apparatus after the second step; and

a fourth step of displaying a second image obtained in the second image taking together with the first image in a predetermined display layout on the display.

In the one aspect of the invention, it is preferably that the image taking apparatus is an eye fundus camera including an optical system and an image pickup device coupled to the control device.

In the one aspect of the invention, it is preferably that the control device can perform image taking in the third step in a state that the first image is displayed on the display and causes the display to display the second image together with the first image immediately after the image taking.

In the one aspect of the invention, it is preferably that the control device controls to image-take one of a left eye and a right eye of the patent in the first step, controls to image-take the other of the left eye and the right eye in the third step, and causes the display to display images of the both eyes in a layout such that a right eye image is displayed in a right side of a left eye image in the fourth step.

In the one aspect of the invention, it is preferably that the control device causes the display to display images of the both eyes in a layout such that a plurality of left eye images are indicated as thumbnail images in a left side on a screen and a plurality of right eye images are indicated as thumbnail images in a right side on the screen.

In the one aspect of the invention, it is preferably that the control device causes the display to display the first image in a position on the display corresponding to an indication position of a fixation light for the eye at the first image taking in the second step and causes the display to display the second image in a position on the display corresponding to an indication position of the fixation light for the eye at the second image taking in the fourth step.

In the one aspect of the invention, it is preferably that the image taking apparatus has a camera main body and the control device causes the display to display the first image in a position on the display corresponding to an image taking position by at least one of panning and tilting of the camera main body at the first image taking in the second step and causes the display to display the second image in a position on the display corresponding to an image taking position by at least one of panning and tilting of the camera main body at the second image taking in the fourth step.

In the above aspect of the invention, it is preferably that the image taking apparatus is an eye fundus image taking apparatus and the control device combines the first image and the second image and causes the display to display a panorama image

According to another aspect of the present invention, there is provided an ophthalmologic apparatus including:

an image taking apparatus for image-taking an eye of a patient;

a display for displaying an image of the eye of the patient, which is image-taken by the image taking apparatus; and

a control device,

wherein the control device executes the steps of;

determining whether the eye image-taken by the image taking apparatus is a left eye or a right eye;

displaying the image obtained in the image taking on the display; and

displaying images of the both eyes on the display in a layout such that a right eye image is displayed in a right side of a left eye image, in accordance with a determination result by the determining step.

In the another aspect of the invention, it is preferably that the image taking apparatus is an eye fundus camera including an optical system and an image pickup device coupled to the control device.

According to further aspect of the present invention, there is provided a method of displaying an ophthalmic image on a display medium, including the steps of:

determining whether an eye image-taken by an image taking apparatus is a left eye or a right eye; and

arranging and indicating images of the both eyes on a display medium in a layout such that a right eye image is displayed in a right side of a left eye image, in accordance with a determination result by the determining step.

In the further aspect of the invention, it is preferably that a plurality of left eye images are indicated as thumbnail images in a left side on a display medium and a plurality of right eye images are indicated as thumbnail images in a right side of the indicated left eye images.

Further objects of the present invention and embodiments thereof become clear in an explanation of the embodiments below.

### BRIEF DESCRIPTION OP THE DRAWINGS

FIG. 1 is a structural view of a first embodiment;

FIG. 2 is an explanatory view of image capture timing by strobe exposure;

FIG. 3 is an explanatory view of a layout of a thumbnail display;

FIG. 4 shows a display example of an eye fundus image on a display;

FIG. 5 is a flow chart for determining an image display position;

FIG. 6 is an explanatory view of a taken image display layout on the display;

FIG. 7 is a front view of the eye fundus image displayed on the display;

FIG. 8 is a structural view of a second embodiment;

FIGS. 9A, 9B and 9C are explanatory views of a taken image display layout according to the second embodiment;

FIG. 10 is a structural view of a third embodiment;

FIG. 11 is an explanatory view of a panorama display layout of a taken image according to the third embodiment;

FIG. 12 is an explanatory view of a conventional image display example; and

FIG. 13 is an explanatory view of another conventional image display example.

### DESCRIPTION OF THE PREFERRED EMBODIMENTS

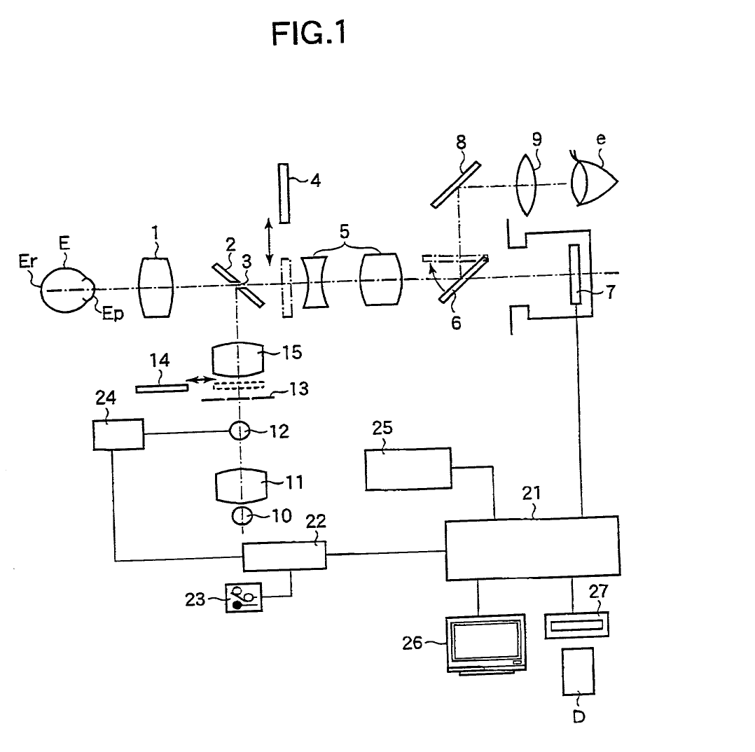

FIG. 1 is a structural view of an eye fundus camera according to a first embodiment. An objective lens 1 is disposed in front of an eye of a patient E. In a back side thereof on an optical path, a hole mirror 2, an image taking diaphragm 3 located in a hole of the hole mirror 2, a barrier filter 4 inserted onto the optical path at photofluorography, an image taking lens 5 in which a portion is movable for focusing, a movable mirror 6, and an image pickup device 7 are arranged. A mirror 8 and an eyepiece 9 are disposed in a reflecting direction of the movable mirror 6. In an incident direction of illumination light to the hole mirror 2, an observation light source 10 such as a halogen lump for emitting visual light, a condenser lens 11, a strobe light source 12 for emitting a flash of the visual light, a diaphragm 13 having a ring-shaped aperture, an exciter filter 14 inserted onto the optical path at photofluorography, and a lens 15 are arranged.

An output of the image pickup device 7 is connected with an image processor 21 including a frame memory. The Image processor 21 is connected with a system controller 22. A right and left eyes switch 23 of the eye fundus camera is connected with the system controller 22. An output of the system controller 22 is connected with the strobe light source 12 through a strobe light emitting controller 24. Also, an input device 25, a display 26 as a display medium, and a memory device 27 are connected with the image processor 21. Note that a computer including a video capture board may be used as an example of the image processor 21. Also, in addition to the display 26, a printer may be provided as a display medium.

An eye fundus image taking optical system is constructed by the objective lens 1, the image taking diaphragm 3, the barrier filter 4, and the image taking lens 5 and an eye fundus image pickup device for image-taking an eye fundus Er of the patient's eye E is constructed by these elements and the image pickup device 7. Also, an observation optical system is constructed by the movable mirror 6, the mirror 8, and the eyepiece 9 and thus an eye fundus image is provided for an observation eye “3”. Further, an illumination optical system is constructed through an optical path from the observation light source 10 to the hole mirror 2.

Next, the operation of the apparatus in a case of photofluorography will be described. First, an operator (photographer or diagnostician) inputs patient information such as the ID number, the name, the date of birth, and the sex of the patient from the input device 25 to the image processor 21 before image taking. Next, the operator causes the patient's eye E to locate in front of the objective lens 1 to perform an alignment for eye fundus image taking.

When the observation light source 10 is turned on, observation light therefrom is condensed by the condenser lens 11, passed through the strobe light source 12, the diaphragm 13, the exciter filter 14 inserted onto the optical path, and the lens 15, reflected to the left side by a mirror portion of the hole mirror 2, and passed through the objective lens 1 and a pupil Ep of the patient's eye E to illuminate the eye fundus Er. Then, an image of the eye fundus Er illuminated with the observation light is again passed through the objective lens 1, the image taking diaphragm 3 in the hole of the hole mirror 2, and the image taking lens 5, reflected upward by the movable mirror 6, further reflected to the right side by the mirror 8, and reaches the observation eye “e” through the eyepiece 9. While viewing the eye fundus image, the operator performs a precision alignment between the patient's eye E and the eye fundus camera, focusing, and a check of an image taking area.

Almost simultaneously with an injection of a fluorescent agent to the patient, the operator pushes a timer switch (not shown) to start time measurement. Then, while observing the patient's eye E, the operator waits for the fluorescent image to appear in the eye fundus. When the fluorescent image is observed, the operator operates an image taking switch (not shown) to perform still image taking after checking that the image taking area, the position, and the focusing are satisfactory. When an input by the image taking switch is detected, the system controller 22 controls to flip the movable mirror 6 such that it is moved outside the optical path. Simultaneously, the system controller 22 controls to insert the barrier filter 4 into the optical path and outputs an image taking start signal to the image processor 21, and thus waits for a light emitting timing signal for the strobe light source 12 from the image processor 21.

Here, the light emitting timing signal by the strobe light source 12 synchronized with the image pickup device 7 will be described. In the case where a video camera is used as the image pickup device 7 and a still image is taken with strobe emitting, when an image with relatively no movement such as an eye fundus image becomes a subject, in order to perform maximum high resolution image taking, generally, a video camera is driven with a frame accumulation mode of a image pickup device such as a CCD.

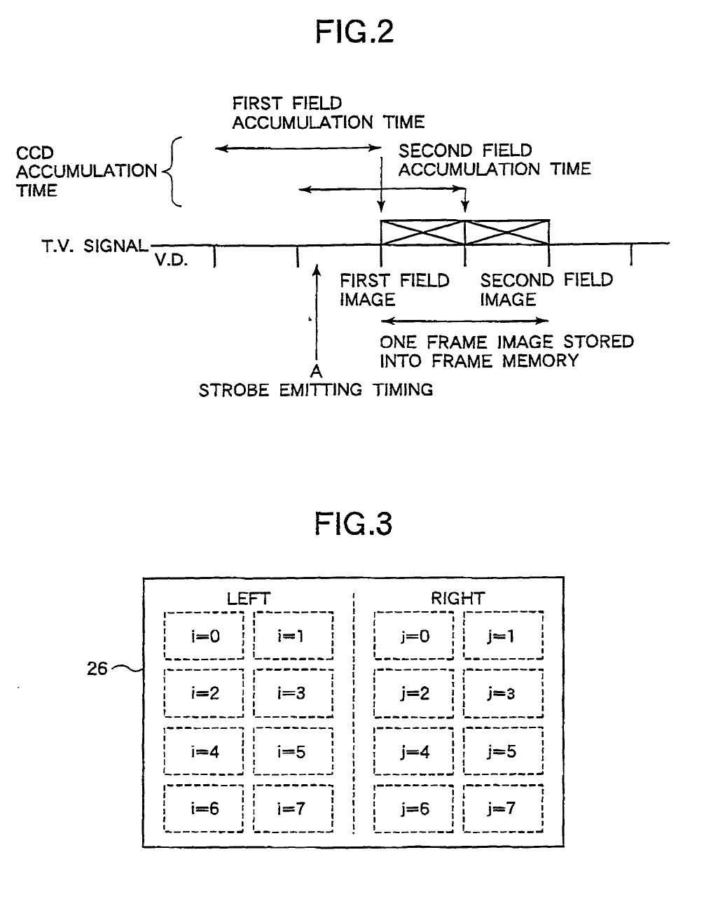

As shown in FIG. 2, in a case of the frame accumulation mode, one frame is composed of two fields, that is, a first field and a second field. A light accumulation period of each field image becomes one frame period immediately before each field image is outputted. That is, the accumulation periods of two fields are shifted by one field with respect to each other. Thus, in order to obtain one frame image with one strobe emitting, it is necessary to cause the strobe light source 12 to emit light for an accumulation period common to two fields composing one frame. The image processor 21 provides instructions to the system controller 22 so as to perform strobe emitting at a timing of “A” shown in FIG. 2.

In the case of a digital camera using the CCD as the image pickup device 7, when the image taking start signal is received from the system controller 22, the image processor 21 outputs an image taking signal to the image pickup device 7. After receiving a light accumulation start signal from the image pickup device 7, the image processor 21 outputs the light emitting timing signal for the strobe light source 12 to the system controller 22.

When the light emitting timing signal for the strobe light source 12 is received from the image processor 21, the system controller 22 transmits a light emitting signal to the strobe light emitting controller 24 without delay to cause the strobe light source 12 to emit light. As in the case of the observation light, light flux emitted from the strobe light source 12 is passed through the ring-shaped aperture of the diaphragm 13, the exciter filter 14 and the lens 15, reflected to the left side by the mirror portion in the periphery of the hole mirror 2, and passed through the objective lens 1 to illuminate the eye fundus Er from the pupil Ep of the patient's eye E. An image of the eye fundus Er thus illuminated is again passed through the objective lens 1, the image taking diaphragm 3 in the hole of the hole mirror 2, and come to a barrier filter 4. The illuminated image passing through the barrier filter 4 is passed through the image taking lens 5 and imaged onto an image pickup surface of the Image pickup device 7. In the case where the image pickup device 7 is a television camera, the image pickup device 7 outputs a television signal to the image processor 21. In the case where the image pickup device 7 is a digital camera, the image pickup device 7 outputs a digital image signal to the Image processor 21.

The image processor 21 synchronizes the television signal or the image signal of one frame outputted from the image pickup device 7 with the strobe emitting and, in case of the television signal, the signal is stored into frame memory after A/D conversion, but, in case of the digital image signal, the signal is stored as it is. Subsequently, the image processor 21 reads image taking information such as left or right eye information from the system controller 22 and uses this information in addition to the above patient information as supplementary information related to the taken image stored in the frame memory. At this time, this supplementary information may be separated from image data and used as text information or can be written into a tag region of an image format. Note that the image taking information such as left and right eyes information can be inputted from the input device 25 to the image processor 21 by the operator.

The image processor 21 writes the image data of the frame memory into an image display memory. In accordance with contents of the supplementary information at this time, an address on an image display memory into which the image data is written is changed. The image data written into the image display memory is D/A-converted and then outputted to the display 26. This is performed immediately after the image taking. Thus, the operator can progress the image taking while checking an image displayed with a predetermined layout based on the image supplementary information. Also, the image processor 21 performs write to or readout from a recording medium capable of storing such as an MO, an MD, a DVD-RAM, a VCR, a hard disk or the like through the memory device 27.

Here, a determination example until the address on the image display memory is determined based on contents of the supplementary information will be described. When image taking of a plurality of images is performed for the same patient as in the case of the photofluorography, thumbnail images are displayed such that a series of taken images can be viewed in a list form. In this embodiment, the display positions of the thumbnail images are determined in accordance with the right and left eyes information included in the above supplementary information.

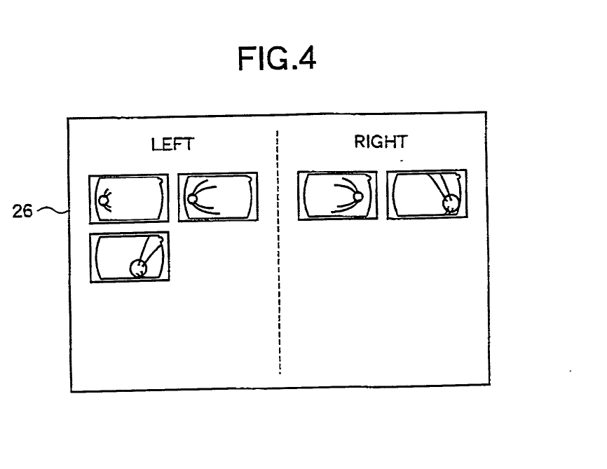

FIG. 3 shows a display layout of the thumbnail images on the display 26. A left side region on a screen is set as a left eye display area LEFT and a right side region thereon is set as a right eye display area RIGHT. In the left side region, a plurality of left eye images are indicated as the thumbnail images. In the right side region, a plurality of right eye images are indicated as the thumbnail images. A variable “i” in the left eye display area LEFT and a variable “j” in the right eye display area RIGHT are parameters indicating display positions and initial values “i” and “j” at start of the image taking are both cleared to be zero lion. FIG. 4 shows an example in the case where thumbnail eye fundus images are displayed on the display 26.

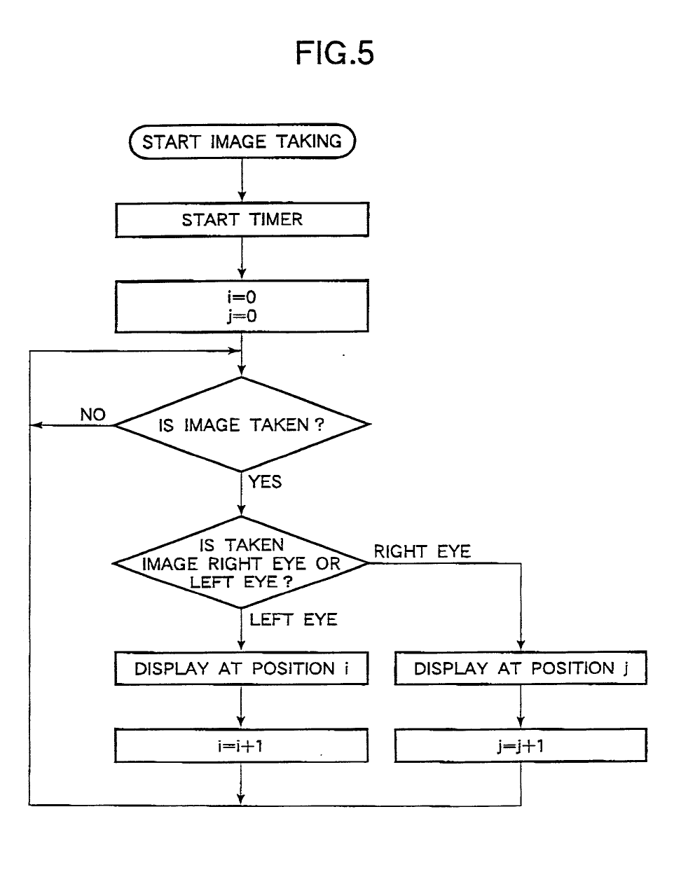

FIG. 5 is a determination flow chart at photofluorography. When a fluorescent timer is actuated to start photofluorography, first, the parameters “i ” and “j ” indicating the positions for displaying thumbnail images are both cleared to be “i”−0 and “j”−0. After image taxing, whether the taken image is the right eye image or the left eye image is determined in accordance with the supplementary information of the image. In the case of the right eye image, the thumbnail image is displayed at the position of “j” and the parameter “j” is incremented by one. Similarly, in the case of the left eye image, the thumbnail image is displayed at the position of “i” and the parameter “i” is incremented by one.

Also, when image taking with one image for each of right and left eyes is performed as in the case of a physical examination, a method of dividing the screen of the display 26 in two regions and enlarging and displaying each image in a displayable range is suitable.

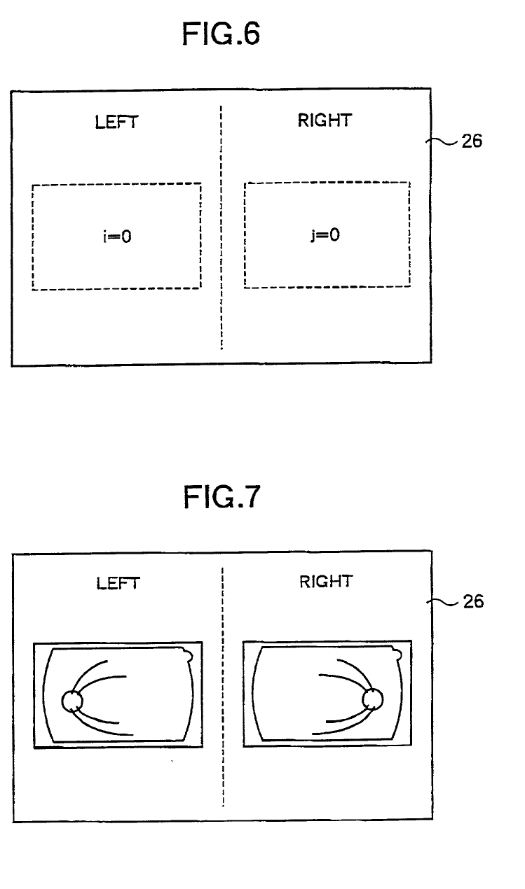

FIG. 6 shows image display positions on the screen of the display 26. The left side region on the screen is the left eye display area LEFT and the right side region thereon is the right eye display area RIGHT. FIG. 7 shows an example in the case where the eye fundus images of the left and right eyes are displayed on these areas. Final images displayed on the display 26 may be printed out to a printer (not shown) in the same layout.

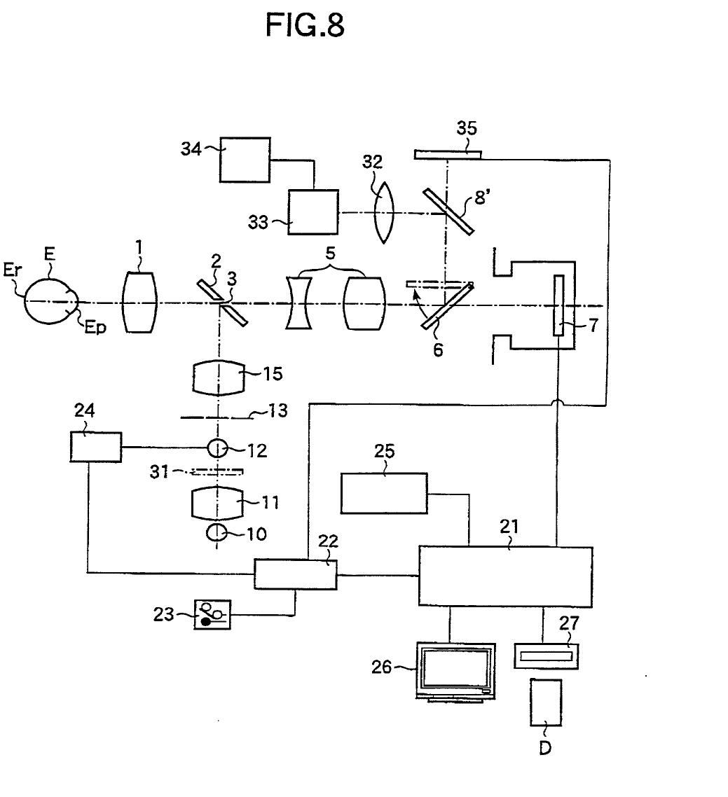

FIG. 8 is a structural view of a second embodiment and shows an example of a non-mydriatic eye fundus camera capable of panorama image taking. This displays the panorama image based on a plurality of eye fundus taken images. The same reference numerals as in FIG. 1 indicate the same members. In the case of image taking using the television camera as an image taking medium, which is recently performed, the amount of image taking light for illuminating the patient's eye E is sufficiently lower than that in conventional film image taking. Thus, even when the non-mydriatic eye fundus camera is used, image taking with a plurality of images can be performed.

Here, a visual light cutting filter 31 is inserted in a position between the condenser lens 11 and the strobe light source 12 of the illumination optical system. Also, a half mirror 8′ is disposed instead of the mirror 8 and a relay lens 32 and a television camera 33 having the sensitivity to an infrared wavelength region are disposed in its reflecting direction. An output of the television camera 33 is connected with a display 34. A fixation light 35 conjugate with the eye fundus Er is disposed in a transmitting direction of the half mirror 8′, and thus a fixation target is indicated for the patient's eye E. An output of the system controller 22 is connected with the fixation light 35.

The fixation light 35 is composed of an LED array arranged in dot matrix form or composed of a back light such as an LED and a dot matrix liquid crystal shutter. The fixation light 35 is controlled such that light is transmitted or not transmitted through a dot in an arbitrary position of two dimensional arrangements, to indicate the fixation target in an arbitrary position with respect to the patient eye's by instructions from the system controller 22. Also, although not shown, an external fixation light may be used as the fixation light independent of the image taking optical system.

When the eye fundus Er of the patient's eye E is observed, light emitted from the observation light source 10 is passed through the visual light cutting filter 31 to become only infrared light. Then, as in the case of the first embodiment, the infrared light is condensed by the condenser lens 11, passed through the strobe light source 12, the diaphragm 13, and the lens 15, reflected to the left side by the mirror portion of the hole mirror 2, and passed through the objective lens 1 and the pupil Ep of the patient's eye. Thus, the eye fundus Er is illuminated as the infrared light.

An image of the eye fundus Er is again passed through the objective lens 1, the image taking diaphragm 3 in the hole of the hole mirror 2, and the image taking lens 5, reflected upward by the movable mirror 6, reflected to the left side by the half mirror 8′, and reaches the television camera 33 through the relay lens 32. The television camera 33 having the sensitivity to the infrared light converts a received eye fundus image into a television signal and outputs it to the display 34.

While viewing the eye fundus image on the display 34, the operator changes the position of the fixation light 35 such that an image taking portion of a target is positioned in the central portion of the screen. Thus, an alignment and a focal adjustment which are required for the eye fundus image taking are performed. Then, the image taking switch is pushed to perform the image taking.

The operation until the image processor 21 causes the frame memory to store image data is performed in the same manner as in the case of the first embodiment. Subsequently, the image processor 21 reads, from the system controller 22, the position of the fixation target indicated from the fixation light 35 to the patient's eye E and uses the fixation target position as the supplementary information of the image as in the case of the first embodiment.

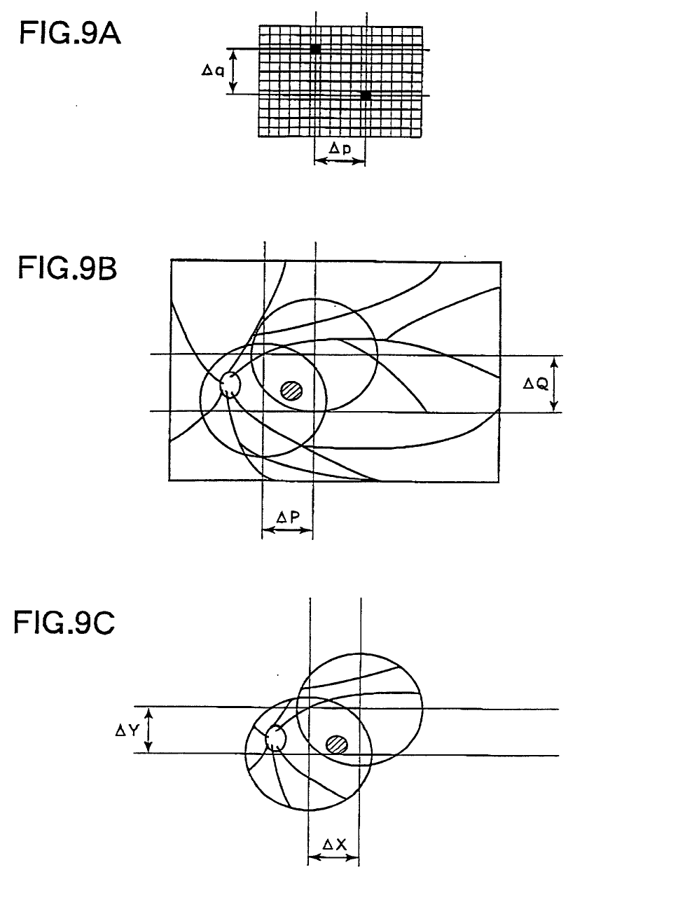

The determination until the address on the image display memory is determined in the case where the position of the fixation light 35 as the supplementary information of the image data is shifted will be described based on FIGS. 9A to 9C. Here, generally, a first taken image indicates the fixation light 35 at a position in which optic disc can be image-taken, and thus image data may be written into substantially the central portion of the upper side on the image display memory. The case where the indication position of the fixation light 35 is changed with respect to second and later taken images will be described.

The fixation light 35 is at a position conjugate with the eye fundus Er. The position of the yellow spot of the eye fundus Er is led based on the indication position of the fixation light 35. When the view angle is considered as a common parameter, the displacement of the fixation light 35 and that of the portion of the taken image can be easily understood. That is, when the displacement is considered by the view angle, although FIG. 9A shows the displacement of the indication position of the fixation light, the displacement (Δp, Δq) of the fixation light 35 corresponds to the displacement (ΔP, ΔQ) of the eye fundus image shown in FIG. 9B. The number of pixels on the image display memory per view angle of one degree is obtained based on an imaging magnification. Thus, the case where the view angle of one degree corresponds to a k-pixel on the image display memory and thus image data is written into the image display memory shown in FIG. 9C is considered.

When the indication position of the fixation light 35 is shifted by the view angle correspondent of (Δp, Δq), the eye fundus image to be image-taken is shifted by (ΔP, ΔQ)=(Δp, Δq) in the view angle. Thus, the displacement (ΔX, ΔY) on the image display memory can be obtained by (ΔX, ΔY)=(kΔP, kΔQ).

Also, when the plurality of eye fundus images are written into one image display memory, it is suitable that an aperture mask in the outline is deleted and thus the images are written. A method of deleting the aperture mask is not described in details here. However, since the position of the aperture mask is generally fixed, this method can be achieved using a method of skipping data in an address corresponding to the position of the aperture mask on the frame memory or a method of skipping a portion in which brightness and color components of image data are substantially zero “0”.

According to the layout based on the position of the fixation light 35, the image displayed immediately after the image taking is checked by the operator. Thus, the remainder in the image taking does not occur. Further, in the panorama image in which respective images are superimposed on each other, the operator can check, during the image taking, whether or not the amount of image taking light is suitable for each image, whether or not the entire amount of image taking light is varied, whether or not flare is entered, or the like. Also, according to a method of determining the address of the image data stored in the image display memory based on the position information of the fixation light 35, since complicated image processing is not required, processing time can be shortened. Note that a final image displayed on the display may be printed out to a printer (not shown) with the same layout.

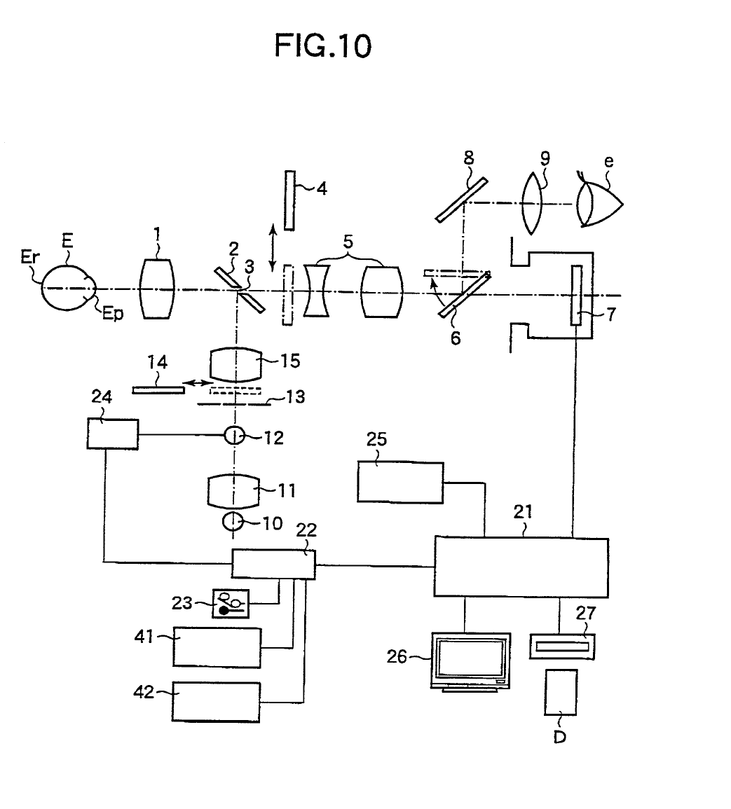

FIG. 10 is a structural view of an eye fundus camera capable of panorama image taking according to a third embodiment. A panorama image is displayed based on a plurality of eye fundus taking images. With respect to the structure of the first embodiment as shown in FIG. 1, an output of a potentiometer 41 for detecting an angle displacement in a position of the eye fundus camera main body by a panning mechanism and an output of a potentiometer 42 for detecting the angle displacement in the position of the eye fundus camera main body by a tilting mechanism are connected with the system controller 22

While observing the eye fundus image through the eyepiece 9, the operator performs tilting operation and panning operation of the eye fundus camera main body such that an image taking portion of a target is positioned in the central portion of the screen. Also, the operator performs an alignment and a focal adjustment, which are required for the eye fundus image taking. Then, the image taking switch (not shown) is pushed to perform the image taking. The operation until the image processor 21 causes the frame memory to store image data is performed in the same manner as in the case of the first embodiment.

After the image data is stored in the frame memory, the image processor 21 reads an displacement in a position of the eye fundus camera main body by the panning mechanism from the output of the potentiometer 41 and an displacement in a position of the eye fundus camera main body by the tilting mechanism from the output of the potentiometer 42, and uses the displacement informations as the supplementary information of the image data stored in the frame memory. The operations of the panning mechanism and the tilting mechanism are performed by taking as an axis the center of the pupil of the patient's eye E. The displacement of an angle in the eye fundus camera main body corresponds to that on the eye fundus. Thus, a write position of the image data into the display memory is determined in the same manner as in the case of the second embodiment.

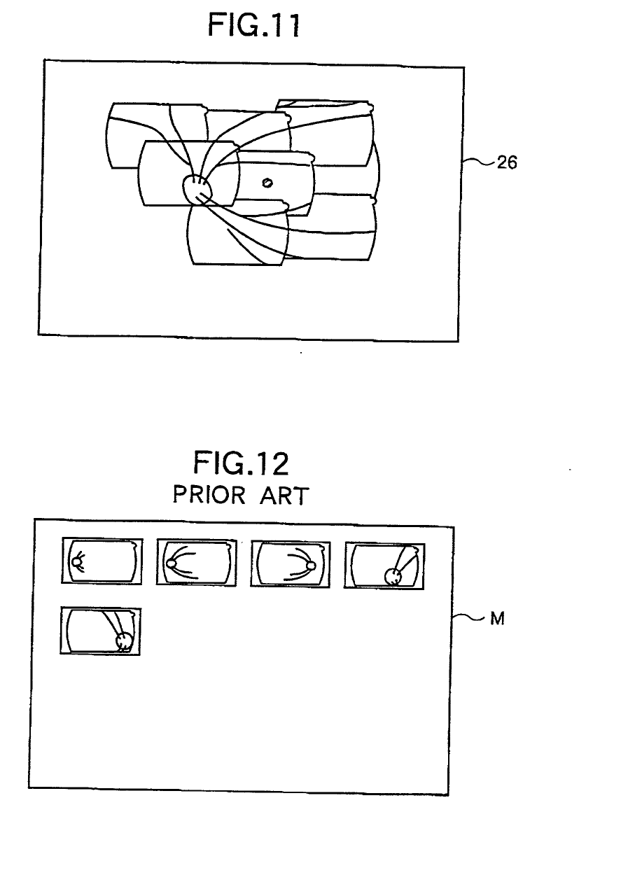

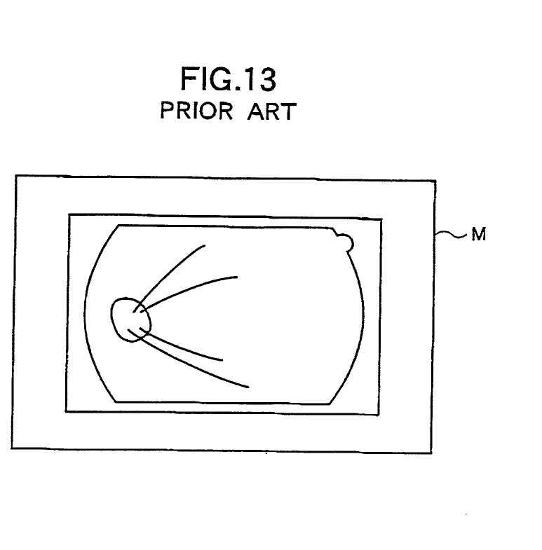

FIG. 11 shows a layout of a panorama image in which a plurality of taken images are combined with each other on the display 26. At the image taking, the panorama image composed of the taken images having combined with each other so far is displayed. After viewing the panorama image and checking the image taking portion, the operator can perform image taking. Then, the taken image with respect to a new portion is immediately combined with the panorama image and thus displayed as a new panorama image on the display. A final image displayed on the display may be printed out to a printer (not shown) in the same layout.

As described above, the taken image is displayed with a display layout having superior usability immediately after the image taking. Thus, the ophthalmologic apparatus which can be used through intuition by an operator and has excellent usability can be realized. Also, that whether or not a necessary image is image-taken immediately after the image taking can be easily determined, a miss such as an image taking omission can be prevented.

What is claimed is:

1. An ophthalmologic apparatus comprising:
an image taking apparatus for image-taking an eye of a patient;
a display for displaying an image of the eye of the patient, which is image-taken by said image taking apparatus; and
a control device,
wherein said control device executes;
a first step of performing the first image taking by said image taking apparatus;
a second step of displaying the first image obtained in the first image taking on said display;
a third step of performing the second image taking by said image taking apparatus after the second step; and
a fourth step of displaying the second image obtained in the second image taking together with the first image in a predetermined display layout on said display.

2. An ophthalmologic apparatus according to claim 1, wherein said image taking apparatus is an eye fundus camera including an optical system and an image pickup device coupled to said control device.

3. An ophthalmologic apparatus according to claim 1, wherein said control device can perform image taking in the third step in a state that the first image is displayed on said display and causes said display to display the second image together with the first image immediately after the image taking.

4. An ophthalmologic apparatus according to claim 1, wherein said control device controls to image-take one of the left eye or the right eye of the patient in the first step, and afterward controls to image-take the other one of the eyes in the third step, and causes said display to display images of the both eyes with a layout such that the right eye image is displayed in a position beside the left eye image in the fourth step.

5. An ophthalmologic apparatus according to claim 1, wherein said control device causes said display to display images of the both eyes in a layout such that a plurality of left eye images are indicated as thumbnail images in a left side on a screen and a plurality of right eye images are indicated as thumbnail images in a right side on the screen.

6. An ophthalmologic apparatus according to claim 1, wherein said control device causes said display to display the first image in a position corresponding to an indication position on said display of a fixation light for the eye at the first image taking in the second step and causes said display to display the second image in a position on said display corresponding to an indication position of the fixation light for the eye at the second image taking in the fourth step.

7. An ophthalmologic apparatus according to claim 1, wherein said image taking apparatus has a camera main body and said control device causes said display to display the first image in a position corresponding to an image taking position by at least one of panning and tilting of said camera main body at the first image taking in the second step and causes said display to display the second image in a position corresponding to an image taking position by at least one of panning and tilting of said camera main body at the second image taking in the fourth step.

8. An ophthalmologic apparatus according to claim 6, wherein said image taking apparatus is an eye fundus image taking apparatus and said control device combines the first image and the second image and causes said display to display a panorama image.

9. An ophthalmologic apparatus comprising:
an image taking apparatus for image-taking an eye of a patient;
a display for displaying an image of the eye of the patient, which is image-taken by said image taking apparatus; and
a control device,
wherein said control device executes the steps of;
determining whether the eye image-taken by said image taking apparatus is a left eye or a right eye;
displaying the image obtained in the image taking on said display; and
displaying images of the both eyes on said display in a layout such that a right eye image is displayed in a right side of a left eye image, in accordance with a determination result by the determining step.

10. An ophthalmologic apparatus according to claim 9, wherein said image taking apparatus is an eye fundus camera which includes an optical system and an image pickup device coupled to said control device.

11. A method of displaying an ophthalmic image on a display medium, comprising the steps of:
determining whether an eye image-taken by an image taking apparatus is a left eye or a right eye; and
arranging and indicating images of the both eyes on a display medium in a layout such that a right eye image is displayed in a right side of a left eye image, in accordance with a determination result by the determining step.

12. A method of displaying an ophthalmic image on a display medium according to claim 11, wherein a plurality of left eye images are indicated as thumbnail images in a left side on the display medium and a plurality of right eye images are indicated as thumbnail images in a right side of the indicated left eye images.
